(19) United States
(12) Patent Application Publication
(10) Pub. No.: US 2026/0273341 A1
(43) Pub. Date: Sep. 17, 2026

(54) EXERCISE MEASUREMENT DEVICE AND SYSTEM

(71) Applicant: VALD PTY LTD, Newstead, Queensland (AU)

(72) Inventors: Samuel JAMES, Newstead (AU); Jason SMITH, Newstead (AU); Luca ROBERTSON, Newstead (AU); Nishant SHRESTHA, Newstead (AU)

(21) Appl. No.: 19/155,222

(22) PCT Filed: Jan. 31, 2024
(86) PCT No.: PCT/AU2024/050056

§ 371 Date: Aug. 8, 2025

(30) Foreign Application Priority Data
Feb. 1, 2023 (AU) 2023900243

(51) Int. Cl. A63B 24/00; A63B 71/06; G01S 17/58; G01S 17/88

(52) U.S. Cl. CPC A63B 24/0062; G01S 17/58; G01S 17/88; A63B 2071/0658; A63B 2220/40; A63B 2220/805; A63B 2225/50

(57) ABSTRACT

A human measurement device, such as for measuring the acceleration, deceleration, speed, and/or timing of an athlete is provided. The device has a body, a power source, a display, and a sensor assembly. The sensor assembly is configurable to measure time of flight to a human longitudinally to a direction of travel of the human. The sensor assembly is also configurable to measure the passing of a human transversely to a direction of travel of the human. The display is a cylindrical LED matrix display that wraps around the body of the device.

### FIELD OF THE INVENTION

The invention relates to a human measurement device and system. In particular, the invention relates, but is not limited, to a portable and wireless electronic measurement device configured to measure speed of a user over a distance.

### BACKGROUND TO THE INVENTION

Reference to background art herein is not to be construed as an admission that such art constitutes common general knowledge.

Often in athletic environments it is desirable to accurately measure the performance of an athlete, whether it is for the purpose of training, competition, rehabilitation, testing, or the like. For example, the VALD™ SmartSpeed™ is a timing gate system whereby a plurality of electronic ‘gates’ are erected that can measure the time it takes for a user of interest, such as an athlete, to pass between. The time that the athlete takes to pass between gates can be measured and then used to assess performance. The manual way to measure distance between gates usually involves the use of a tape measure, which is time consuming to set up particularly in more rugged terrains and the manual set up can be cumbersome and prone to inconsistencies and human error. Additionally, users are required to walk through the gates along an intended trajectory in a pre-defined order during setup in order for the software system to understand the order of gates.

Such timing gates only measure speed over a discrete distance that is determined by the spacing between gates. It is not possible to measure acceleration or deceleration that may occur before, after, or between gates. A hand held speed gun may be used for this purpose, but it is a separate unrelated device that not only requires additional equipment and cost, but it also requires additional setup and handling. Additionally, its recordings are separate and unsynchronised from the timing gates measurements.

Furthermore, such systems do not display, on the device itself, significant meaningful information. At best, a colour may be illuminated with detailed information having to be displayed on an external display device which is typically an optional accessory. Such displays are typically planar meaning their results are only easily readable from a narrow range of viewpoints which is often undesirable and its visibility can be affected by sun glare on a sunny day. To enable both an athlete and trainer/practitioner to be able to immediately see the result, it is often necessary for there to be two or more displays.

### OBJECT OF THE INVENTION

It is an aim of this invention to provide a human measurement device and system which overcomes or ameliorates one or more of the disadvantages or problems described above, or which at least provides a useful alternative.

Other preferred objects of the present invention will become apparent from the following description.

### SUMMARY OF INVENTION

In one form, although it need not be the only or indeed the broadest form there is provided, a human measurement device comprising:
a body;
a power source contained within the body;
a display integrated with the body; and
a sensor assembly configurable to measure time of flight to a human longitudinally to a direction of travel of the human and passing of a human transversely to a direction of travel of the human.

The sensor assembly may comprise a time of flight sensor configurable to measure both longitudinally and transversely to a direction of travel of a human. The sensor assembly may comprise a LIDAR sensor. The body may comprise a window for a laser projection from the LIDAR sensor. A single LIDAR sensor may be configured to measure time of flight of a human longitudinally to a direction of travel of the human and passing of a human transversely to a direction of travel of the human. The LIDAR sensor may have a time of flight range of at least 4 metres, preferably of at least 8 metres, and even more preferably of at least 10 m. Where the user is an athlete, when measuring time of flight of an athlete longitudinally to a direction of travel of the athlete, the LIDAR sensor may measure acceleration and deceleration. When measuring the passing of an athlete transversely to a direction of travel of the athlete, the LIDAR sensor may determine when the athlete passes through a gate. The gate may be defined between the LIDAR sensor and a reflector spaced apart from the LIDAR sensor.

The display may wraparound the body. The body may be substantially cylindrical. The display may be curved. The display may extend circumferentially around at least a substantial portion of the cylindrical body. The display may comprise an array of LEDs. The display may be configured to display characters and/or visual indicators such as icons. The display may be configured to display strings of characters in a rotating manner. The array of LEDs may comprise a plurality of panels, each panel comprising a plurality of LEDs thereon. The plurality of panels may collectively form a cylindrical LED array display.

The power source may comprise a rechargeable battery assembly. The battery assembly may be removable. The battery assembly may be contained in the body by a retention mechanism. The retention mechanism may comprise battery release buttons which can be actuated to allow removal of the battery assembly from the body.

The human measurement device may be configured to communicate with one or more further human measurement devices. The communication between human measurement devices may be “hubless”, with communication between the human measurement devices being without a dedicated network hub device. The human measurement device may be configured to determine its distance from one or more further human measurement devices or alternatively determine its relative position to two or more further human measurement devices. The relative position may comprise 3D coordinates. The measurement of distance and/or position between human measurement devices may be automatic.

In another form, there is a human measurement system comprising:
a primary human measurement device;
one or more further human measurement devices in communication with the primary human measurement device;
wherein a distance between the primary human measurement device and each of the human measurement devices is determined automatically.

The primary human measurement device and one or more further human measurement devices may be the same. The primary human measurement device and one or more further human measurement devices are preferably human measurement devices as described herein. The communication between human measurement devices may be “hubless”. The communication may comprise an Ultra Wideband (UWB) star network.

The human measurement system may further comprise one or more reflectors. Each reflector may be configured to form a gate between itself and a human measurement device.

The human measurement system may further comprise an external device. The external device may comprise a tablet, smart phone, laptop, TV, or the like. The external device may be in communication with one or more of the human measurement devices.

Further features and advantages of the present invention will become apparent from the following detailed description.

### BRIEF DESCRIPTION OF THE DRAWINGS

By way of example only, preferred embodiments of the invention will be described more fully hereinafter with reference to the accompanying figures, wherein:

FIG. 1 illustrates a perspective view of a human measurement device;

FIG. 2 illustrates a top-down diagrammatic view of a plurality of human measurement devices measuring perpendicularly to a direction of travel of the user;

FIG. 3 illustrates a top-down diagrammatic view of a human measurement device measuring longitudinally to a direction of travel of the user;

FIG. 4 illustrates a side-on view of a human measurement device having an LED array display;

FIG. 5 illustrates a side-on view of the other side of the human measurement device having an LED array display illustrated in FIG. 4;

FIG. 6 illustrates an LED array display in isolation; and

FIG. 7 illustrates the LED array display in use.

### DETAILED DESCRIPTION OF THE DRAWINGS

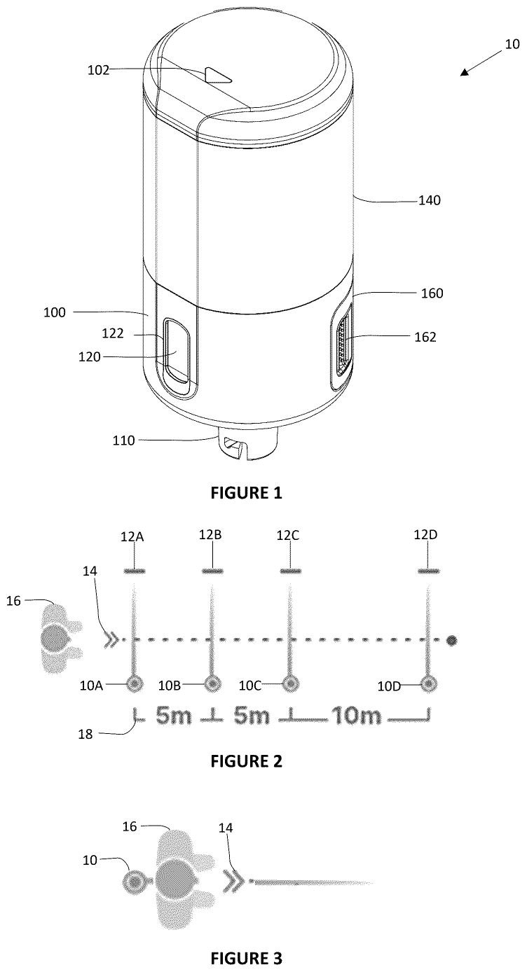

FIG. 1 illustrates a human measurement device 10 comprising a body 100, a mount 110, a sensor assembly 120, and a display 140. The body 100 is substantially cylindrical with the mount 110 located at one end thereof. The mount 110 in the illustrated embodiment is a bayonet style mount, configured to allow the human measurement device 10 to be removably attached to a support such as a tripod or stake, or the like. A plurality of human measurement devices 10 is able to communicate with each other without the need for an external device such as, for example, a hub. This reduces the number of devices required and reduces set up time.

The sensor assembly 120 comprises a window 122 in the body 100 through which a time of flight sensor can see. In a preferred form, the time of flight sensor comprises a laser projection from a LIDAR sensor contained therein can pass. Indicia 102 on the top of the body 100 indicates the direction in which the LIDAR sensor is oriented. The LIDAR preferably has a time of flight range of at least 10 m.

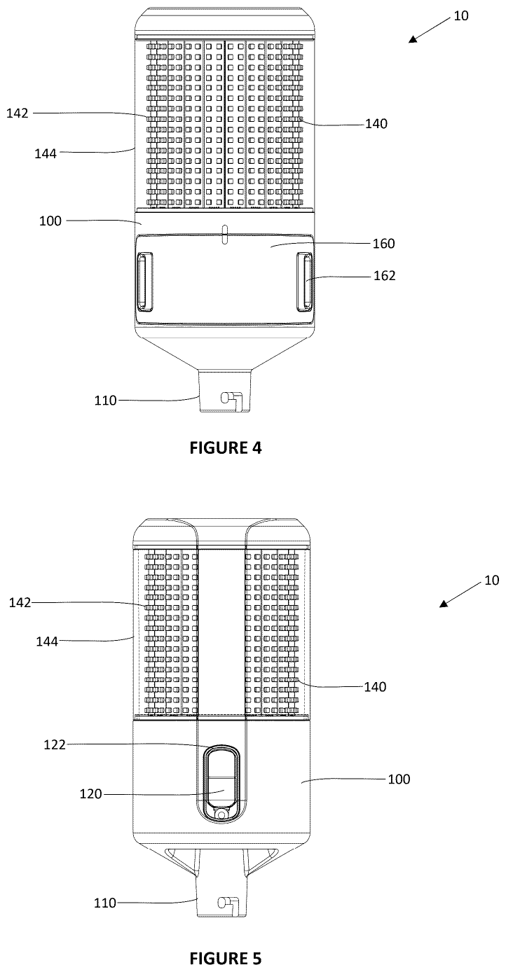

The display 140 is integrated with the body 100. In preferred forms it is curved and extends circumferentially around at least a substantial portion of the cylindrical body 100. The display may comprise any suitable display technology including, for example, an array of LEDs (e.g. see FIGS. 4 to 7), an LCD, an OLED display, or the like. An array of LEDs has been found to provide optimal brightness and visibility in outdoor settings even in full sun. The human measurement device 10 may comprise a light sensor and adjust the brightness of the display 140 according to ambient light levels. The light sensor may be a photoelectric sensor, preferably mounted in an upper surface of the body 100 such as inside the indicia 102. This is a flexible feature designed to allow adjustment of brightness dependent on the use of the device, which is to ensure that the human measurement device 10 is easily visible and legible in outdoor settings across distances, especially during days with full sun light, and also suitable for use in lower light settings, such as indoors. It is preferred that the display wraparound the body 100, as illustrated, to maximise display size without unnecessarily increasing the bulk, and hence decreasing portability, of the device 10. Relevant information regarding the user, their performance, navigation, the configuration of the device, and the like can be shown on the display 140. This can include real-time results display and indications such as instantaneously displaying ‘PB’ for a personal best time being recorded.

The human measurement device 10 comprises a power supply in the form of a rechargeable battery assembly 160. The battery assembly 160 may be entirely internal and charged in device 10 or, more preferably and as illustrated, the battery assembly 160 may be removed allowing swapping of batteries as needed. The battery assembly 160 is held inside the body 100 by a suitable retention mechanism and, in the illustrated embodiment, can be released by actuating battery release buttons 162. Once the battery release 162 has been actuated the battery assembly 160 can be removed from the body 100 and replaced with another battery. This allows significantly longer usage time in the field than usual integrated battery solutions. An additional benefit is that the devices 10 can be left in situation for a long time, even permanently if desired, with batteries being swapped out when needed, avoiding the requirement for the devices 10 to be taken to a power source every time the battery runs low or flat.

FIG. 2 illustrates a usage case of the human measurement device 10 comprising a plurality of human measurement devices 10A-10D with corresponding reflectors 12A-12D. The sensor assembly 120 can be directed towards the reflectors 12A-12D to form gates. The laser projection of the LIDAR sensor of each sensor assembly 120 may be utilised to form a gate between each human measurement devices 10A-10D and its corresponding reflector 12A-12D. It is possible to use LIDAR from the sensor assembly 120 to form a virtual gate without the need for a reflector. However, the reflector improves outdoor detection performance and provides a physical limit to each gate.

In the configuration illustrated in FIG. 2 the human measurement devices 10 measure perpendicularly to a direction of travel 14 of a user 16. As the user 16 passes through each gate there is a disruption to a laser signal transmitted from sensor assembly 120. The timestamp of this disruption, and ensuing laser signal disruptions, are recorded and converted into time metrics. Time metrics can be displayed to the user in real-time on the displays 140 of the human measurement devices 10A-10D or, more comprehensively, on an external device such as a smartphone or tablet. It should be appreciated, however, that other external devices could be utilised such as, for example, a laptop computer. For the illustrated example, a user may measure and see five separate data readings as follows:

| | | | | |
|---|---|---|---|---|
| 1) | Gate 12A-12B: | 0-5 | m | split time |
| 2) | Gate 12B-12C: | 5-10 | m | split time |
| 3) | Gate 12C-12D: | 10-20 | m | split time |
| 4) | Gate 12A-12C: | 0-10 | m | cumulative time |
| 5) | Gate 12A to 12D: | 0-20 | m | cumulative time (total time) |

This data may be stored locally on one or more of the human measurement devices 10, on the external device (not shown), and/or synchronised with a cloud data storage platform.

The human measurement devices 10A-10D are configured to communicate with each other without need for an external device such as, for example, a hub. It may, however, be preferable to have an external device, such as a tablet or laptop computer, in communication with at least one of the human measurement devices 10 for configuration, status updates, result recordal, result analysis, and the like.

The human measurement devices 10A-10D are arranged at distance intervals 18 of interest. The distance intervals 18 can be varied according to the type of exercise or measurement being taken. In prior art devices, the distance intervals 18 would have to be measured manually, usually using a tape measure, which was cumbersome, time consuming, and prone to human error. Local positioning of the devices 10 can be ascertained use a star network. This may be, for example, mesh network or star Network based on a UWB Institute of Electrical and Electronic Engineers (IEEE) technical standard 802.15.4. One of a plurality of human measurement devices 10 may be designated as a primary device. The primary device can effectively replace a hub such that the human measurement devices can communicate in a “hubless” manner. With two human measurement devices 10 a 2D distance can be obtained between the primary device and the other device. With three or more human measurement devices 10 3D coordinates between all devices can be ascertained. In either case, the distance between devices 10 can be measured quickly in an automatic manner. These distance measurements, derived from the devices 10 automatically, can assist with detecting gate patterns. For example, where a gate pattern matches a pre-defined drill type the supporting mobile application can utilise the measured distances between devices 10 to automatically predict a desired drill type.

FIG. 3 illustrates a usage case of the human measurement device 10 wherein a single device 10 is arranged to measure longitudinally to the direction of travel 14 of the user 16. As illustrated the device 10 is located behind the user 16 with its LIDAR directed to measure the user accelerating away from the device 10. It should be appreciated that the device 10, or indeed a further device 10, could be arranged to measure the user 16 approaching (e.g. to measure deceleration after a sprint). In addition to being able to measure acceleration and deceleration, the LIDAR may be configured to measure turn metrics such as depth of turn and time of turn. It should also be appreciated that with sufficient devices the FIG. 2 and FIG. 3 usage cases may be able to be combined to collect both longitudinal and transverse measurement data simultaneously in a synchronised manner allowing direct comparison and analysis of both sets of measurements together.

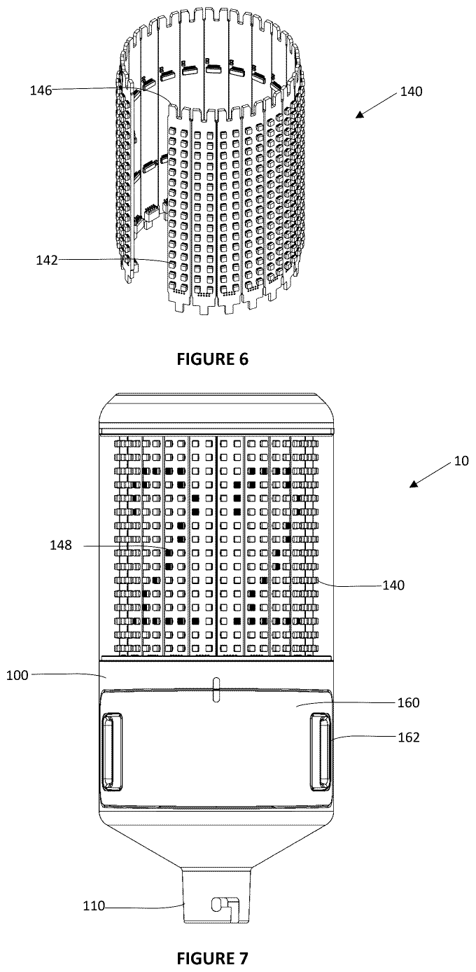

FIGS. 4 and 5 illustrate the human measurement device 10 with a display 140 in the form of an array of LEDs 142 protected by a transparent cover or lens 144 which sits substantially flush with the body 100 of the device 10. As can be seen more clearly in FIG. 6, the LED array display 140 comprises a plurality of panels, in the form of elongate printed circuit boards (PCBs) 146, each having a plurality of the LEDs 142 that form a subset of the display 140. When combined, the LED PCBs 146 collectively form a cylindrical LED array display 140. In the illustrated embodiment there are 20 panels each having two columns of 16 LEDs which, when combined, provides a total display resolution of 40×16 pixels. The number of LEDs can be varied to increase or decrease the resolution.

Characters, such as alphanumeric characters, can be displayed on the LED array display 140. FIG. 7 illustrates the LED array display 140 in use displaying the number ‘22’ with illuminated LEDs 148. Characters may be displayed in a rotating manner, traversing around the display 140, for 360° visibility. The addition of such a display 140 that is able to convey meaningful information, rather than just a flash or colour change, allows removal of the need for an external device (e.g. external display or hub) which further simplifies the system.

Advantageously, the human measurement device 10 provides a portable system that requires fewer different types of devices to perform both transverse and perpendicular gated subject tracking and longitudinal continuous subject tracking. The local positioning system between devices 10 allows accurate and quick determination of the distances 18 between devices 10. Furthermore, it is no longer a requirement for a user to walk a gate trajectory to set up the devices 10 because they already know what order they are in. These improvements not only significantly reduce setup time, but also reduce errors.

The integrated wraparound display 140 can provide information as to which user is next to be measured but showing their display name prior to commencement of the measurement. This prevents the need for users to have to return to a fixed schedule to see when they are scheduled to be tested. The display may also provide device status information such as a warning when the batteries are getting low, avoiding unexpected disruptions that can otherwise occur when a battery runs out of power. The display 140 also provide guidance information, ensuring users know what drill they are performing which significantly reduces the likelihood of users wasting the time and effort or everyone when they make a mistake with respect to the drill they are meant to be performing. The use of an LED array is considered advantageous as high powered LEDs can be used which have a brightness that exceeds that presently available in many other types of displays. This enables the display 140 to be readable over long distances and in environments with high ambient lighting such as when the display 140 is in direct sunlight. The addition of an ambient light sensor allows the brightness of the LEDs to be adjusted as needed in lower light environments, such as when being used indoors.

The integrated LIDAR allows continuous measurement of acceleration and/or deceleration of a user rather than being inferred by discrete datapoints from gate data. A rate of velocity development can be charted for better analysis or performance and skill of an athlete, or the like. Furthermore, it allows a single tool to capture both speed and acceleration data.

A further advantage of the device 10 is that its batteries are easily removable allowing hot swapping of batteries in the field. This is particularly helpful in outdoor settings where batteries can deteriorate quicker due to prolonged exposure to the elements. This further increases the durability and useful life of the devices 10 as it avoids the need for devices 10 to be retired or refurbished upon battery failure. It also allow the devices 10 to remain in situation during a battery swap, avoiding device 10 movements that may affect measurement accuracy without reconfiguration or recalibration.

Although the invention is primarily described with reference to an athlete, it should be appreciated that it may be utilised in relation to other applications such as, for example, measuring and monitoring health and rehabilitation of a patient.

In this specification, adjectives such as first and second, left and right, top and bottom, and the like may be used solely to distinguish one element or action from another element or action without necessarily requiring or implying any actual relationship or order. Where the context permits, reference to an integer or a component or step (or the like) is not to be interpreted as being limited to only one of that integer, component, or step, but rather could be one or more of that integer, component, or step etc.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. The invention is intended to embrace all alternatives, modifications, and variations of the present invention that have been discussed herein, and other embodiments that fall within the spirit and scope of the above described invention.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

1. A human measurement device comprising:
a body;
a power source contained within the body;
a display integrated with the body; and
a sensor assembly configurable to measure time of flight to a human longitudinally to a direction of travel of the human and passing of a human transversely to a direction of travel of the human.

2. The human measurement device of claim 1, wherein the sensor assembly comprises a time of flight sensor configurable to measure both longitudinally and transversely to a direction of travel of a human.

3. The human measurement device of claim 1, wherein the sensor assembly comprises a Light Detection and Ranging (LIDAR) sensor.

4. The human measurement device of claim 1, wherein a single LIDAR sensor is configured to measure time of flight of a human longitudinally to a direction of travel of the human and passing of a human transversely to a direction of travel of the human.

5. The human measurement device of claim 3, wherein when measuring time of flight of an athlete longitudinally to a direction of travel of the athlete, the LIDAR sensor is configured to measure acceleration and deceleration of the athlete.

6. The human measurement device of claim 3, wherein when measuring the passing of an athlete transversely to a direction of travel of the athlete, the LIDAR sensor is configured to determine when the athlete passes through a gate.

7. The human measurement device of claim 1, wherein the body is substantially cylindrical and the display wraps around the body.

8. The human measurement device of claim 1, wherein the display comprises an array of Light Emitting Diodes (LEDs).

9. The human measurement device of claim 1, wherein the power source comprises a removable rechargeable battery assembly that is contained in the body by a retention mechanism.

10. The human measurement device of claim 9, wherein the retention mechanism comprises release buttons which can be actuated to allow removal of the battery assembly from the body.

11. The human measurement device of claim 1, wherein the human measurement device is configured to communicate with one or more further human measurement devices.

12. The human measurement device of claim 11, wherein the communication between human measurement devices is hubless, being communication between the human measurement devices being without a dedicated network hub device.

13. The human measurement device of claim 1, wherein the human measurement device is configured to determine its distance from one or more further human measurement devices.

14. The human measurement device of claim 1, wherein the human measurement device is configured to determine its relative position to two or more further human measurement devices.

15. The human measurement device of claim 14, wherein the relative position comprises 3D coordinates.

16. The human measurement device of claim 13 wherein the measurement of one or more of distance position between human measurement devices and position between human measurement devices is automatic.

17. A human measurement system comprising:
a primary human measurement device, the primary human measurement device being a human measurement device according to claim 1; and
one or more further human measurement devices in communication with the primary human measurement device;
wherein a distance between the primary human measurement device and each of the human measurement devices is determined automatically.

18. The human measurement system of claim 17, wherein the one or more further human measurement devices are human measurement devices according to any one of claims 1 to 16.

19. The human measurement system of claim 17, wherein the communication comprises an Ultra Wideband (UWB) star network.

20. The human measurement system of claim 17, further comprising one or more reflectors configured to form a gate between itself and a human measurement device.
